SEQUENCE LISTING (70 sequences)

SEQ ID NO: 1 - DNA, 852 bp, Homo sapiens
  CDS (1)..(852)
atgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatga

SEQ ID NO: 2 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 3 - DNA, 1053 bp, Homo sapiens
  CDS (1)..(1053)
atgctgggctcagtgaagatggaggcccatgacctggccgagtggagctactacccggaggcgggcgaggtctactcgccggtgaccccagtgcccaccatggcccccctcaactcctacatgaccctgaatcctctaagctctccctatccccctggggggctccctgcctccccactgccctcaggacccctggcacccccagcacctgcagcccccctggggcccactttcccaggcctgggtgtcagcggtggcagcagcagctccgggtacggggccccgggtcctgggctggtgcacgggaaggagatgccgaaggggtatcggcggcccctggcacacgccaagccaccgtattcctatatctcactcatcaccatggccatccagcaggcgccgggcaagatgctgaccttgagtgaaatctaccagtggatcatggacctcttcccttactaccgggagaatcagcagcgctggcagaactccattcgccactcgctgtctttcaacgactgcttcgtcaaggtggcgcgttccccagacaagcctggcaagggctcctactgggccctacaccccagctcagggaacatgtttgagaatggctgctacctgcgccgccagaaacgcttcaagctggaggagaaggtgaaaaaagggggcagcggggctgccaccaccaccaggaacgggacagggtctgctgcctcgaccaccacccccgcggccacagtcacctccccgccccagcccccgcctccagcccctgagcctgaggcccagggcggggaagatgtgggggctctggactgtggctcacccgcttcctccacaccctatttcactggcctggagctcccaggggagctgaagctggacgcgccctacaacttcaaccaccctttctccatcaacaacctaatgtcagaacagacaccagcacctcccaaactggacgtggggtttgggggctacggggctgaaggtggggagcctggagtctactaccagggcctctattcccgctctttgcttaatgcatcctag

SEQ ID NO: 4 - protein, 350 aa, Homo sapiens
MLGSVKMEAHDLAEWSYYPEAGEVYSPVTPVPTMAPLNSYMTLNPLSSPYPPGGLPASPLPSGPLAPPAPAAPLGPTFPGLGVSGGSSSSGYGAPGPGLVHGKEMPKGYRRPLAHAKPPYSYISLITMAIQQAPGKMLTLSEIYQWIMDLFPYYRENQQRWQNSIRHSLSFNDCFVKVARSPDKPGKGSYWALHPSSGNMFENGCYLRRQKRFKLEEKVKKGGSGAATTTRNGTGSAASTTTPAATVTSPPQPPPPAPEPEAQGGEDVGALDCGSPASSTPYFTGLELPGELKLDAPYNFNHPFSINNLMSEQTPAPPKLDVGFGGYGAEGGEPGVYYQGLYSRSLLNAS

SEQ ID NO: 5 - DNA, 1704 bp, Homo sapiens
  CDS (1)..(1704)
atggcgcagcccccccggctgagccgctctggtgcctcctcactttgggacccagcttctcctgctcccacctctggccccaggcctcggctttgggagggtcaagatgtgctggccagatggactgatgggctgctatacttgggtaccatcaaaaaggtggacagtgctagggaggtgtgtctggtccagtttgaggatgattcgcagtttctggttctatggaaagacattagccctgctgccctccctggagaggaactcctctgttgtgtctgtcgctctgagactgtggtccctgggaaccggttggtcagctgtgagaagtgtcgccatgcttatcaccaggactgccatgttcccagggctccagcccctggagagggagagggcacatcctgggtatgccgccagtgtgtctttgcgatcgccaccaagaggggaggtgccctgaagaagggcccctatgcccgggccatgctgggtatgaagctttctctgccatatggactgaaggggctggactgggatgctggacatctgagcaaccgacagcagagttactgttactgtggtggccctggggagtggaacctgaaaatgctgcagtgccggagctgcctgcagtggttccatgaggcctgcacccagtgtctgagcaagcccctcctctatggggacaggttctatgaatttgaatgctgtgtgtgtcgcgggggccctgagaaagtccggagactacagcttcgctgggtggatgtggcccatcttgtcctgtatcacctcagtgtttgctgtaagaagaaatactttgattttgatcgtgagatcctccccttcacttctgagaattgggacagtttgctcctgggggagctttcagacacccccaaaggagaacgttcttccaagctcctctctgctcttaacagccacaaggaccgtttcatttcagggagagagattaagaagaggaaatgtttgtttggtctccatgctcggatgcctccccctgtggagccccctactggagatggagcactcaccagcttcccttcagggcagggccctgggggaggggtctcacgtcccctggggaagcgccggaggccggagccagagcccctgaggaggaggcagaaggggaaagtggaggagctggggccaccctcagcagtgcgcaatcagcccgagccccaggagcagagggagcgggctcatctgcagagggcactgcaggcctcagtgtctccaccatcccccagccctaaccagagttaccagggcagcagcggctacaacttccggcccacagatgcccgctgcctgcccagcagccccatccggatgtttgcttccttccacccttctgccagcaccgcagggacctctggggacagtggacccccagacaggtcacccctggaacttcacattggtttccccacagacatccctaaaagtgccccccactcgatgactgcctcatcttcctcagtttcatccccatccccaggtcttcctagacgctcagcacccccttctcccctgtgccgtagtttgtctcctgggactgggggaggagtccgaggtggggttggttacctgtcccgaggggaccctgtccgggtccttgctcggagagtacggcctgatggctctgtgcagtacctggttgagtggggaggagggggcatcttctga

SEQ ID NO: 6 - protein, 567 aa, Homo sapiens
MAQPPRLSRSGASSLWDPASPAPTSGPRPRLWEGQDVLARWTDGLLYLGTIKKVDSAREVCLVQFEDDSQFLVLWKDISPAALPGEELLCCVCRSETVVPGNRLVSCEKCRHAYHQDCHVPRAPAPGEGEGTSWVCRQCVFAIATKRGGALKKGPYARAMLGMKLSLPYGLKGLDWDAGHLSNRQQSYCYCGGPGEWNLKMLQCRSCLQWFHEACTQCLSKPLLYGDRFYEFECCVCRGGPEKVRRLQLRWVDVAHLVLYHLSVCCKKKYFDFDREILPFTSENWDSLLLGELSDTPKGERSSKLLSALNSHKDRFISGREIKKRKCLFGLHARMPPPVEPPTGDGALTSFPSGQGPGGGVSRPLGKRRRPEPEPLRRRQKGKVEELGPPSAVRNQPEPQEQRERAHLQRALQASVSPPSPSPNQSYQGSSGYNFRPTDARCLPSSPIRMFASFHPSASTAGTSGDSGPPDRSPLELHIGFPTDIPKSAPHSMTASSSSVSSPSPGLPRRSAPPSPLCRSLSPGTGGGVRGGVGYLSRGDPVRVLARRVRPDGSVQYLVEWGGGGIF

SEQ ID NO: 7 - DNA, 723 bp, Homo sapiens
  CDS (1)..(723)
atgacctctttgccctgccccctccccggccgggacgcctccaaagctgtcttcccagacctcgcccctgtcccgtcggtagcggctgcctacccgcttggcttgtcccctacaaccgcagcctcccccaatttgtcctactccaggccgtatggccacctcctgtcttacccctacaccgagccagcgaaccccggagactcctacctgtcctgccagcaacccgcggcgctctctcagcccctctgcggacctgcagagcaccctcaggaactcgaggcagactcggagaagccgcggctgtccccggaaccctccgagcggcgccctcaggcccccgccaaaaagctccgcaagccgaggaccatctactccagcctgcagctgcagcacctaaaccagcgtttccagcacacgcagtacctggcgctgcccgagagggcccagctggcagcgcagctcggcctcacccagacccaggtaaagatctggtttcagaacaaacgctccaagtataagaagctcctgaagcagaattctggggggcaggaaggggacttccctgggaggaccttctctgtgtctccctgctccccacccctcccctccctctgggatctacccaaggcagggaccctgcccaccagtggctatggcaacagctttggagcctggtatcagcatcactcctcagatgtcctggcttcgcctcagatgatgtga

SEQ ID NO: 8 - protein, 240 aa, Homo sapiens
MTSLPCPLPGRDASKAVFPDLAPVPSVAAAYPLGLSPTTAASPNLSYSRPYGHLLSYPYTEPANPGDSYLSCQQPAALSQPLCGPAEHPQELEADSEKPRLSPEPSERRPQAPAKKLRKPRTIYSSLQLQHLNQRFQHTQYLALPERAQLAAQLGLTQTQVKIWFQNKRSKYKKLLKQNSGGQEGDFPGRTFSVSPCSPPLPSLWDLPKAGTLPTSGYGNSFGAWYQHHSSDVLASPQMM

SEQ ID NO: 9 - DNA, 2004 bp, Homo sapiens
  CDS (1)..(2004)
atgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtacagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcagagcctccccctctctccttggccggcccacaccctggaatgggagacgcatcgaatcacatgggacagatgtaa

SEQ ID NO: 10 - protein, 667 aa, Homo sapiens
MHHQQRMAALGTDKELSDLLDFSAMFSPPVSSGKNGPTSLASGHFTGSNVEDRSSSGSWGNGGHPSPSRNYGDGTPYDHMTSRDLGSHDNLSPPFVNSRIQSKTERGSYSSYGRESNLQGCHQQSLLGGDMDMGNPGTLSPTKPGSQYYQYSSNNPRRRPLHSSAMEVQTKKVRKVPPGLPSSVYAPSASTADYNRDSPGYPSSKPATSTFPSSFFMQDGHHSSDPWSSSSGMNQPGYAGMLGNSSHIPQSSSYCSLHPHERLSYPSHSSADINSSLPPMSTFHRSGTNHYSTSSCTPPANGTDSIMANRGSGAAGSSQTGDALGKALASIYSPDHTNNSFSSNPSTPVGSPPSLSAGTAVWSRNGGQASSSPNYEGPLHSLQSRIEDRLERLDDAIHVLRNHAVGPSTAMPGGHGDMHGIIGPSHNGAMGGLGSGYGTGLLSANRHSLMVGTHREDGVALRGSHSLLPNQVPVPQLPVQSATSPDLNPPQDPYRGMPPGLQGQSVSSGSSEIKSDDEGDENLQDTKSSEDKKLDDDKKDIKSITSNNDDEDLTPEQKAEREKERRMANNARERLRVRDINEAFKELGRMVQLHLKSDKPQTKLLILHQAVAVILSLEQQVRERNLNPKAACLKRREEEKVSSEPPPLSLAGPHPGMGDASNHMGQM

SEQ ID NO: 11 - DNA, 2085 bp, Homo sapiens
  CDS (1)..(2085)
atgccggagttcctggaagacccctcggtcctgacaaaagacaagttgaagagtgagttggtcgccaacaatgtgacgctgccggccggggagcagcgcaaagacgtgtacgtccagctctacctgcagcacctcacggctcgcaaccggccgccgctccccgccggcaccaacagcaaggggcccccggacttctccagtgacgaagagcgcgagcccaccccggtcctcggctctggggccgccgccgcgggccggagccgagcagccgtcggcaggaaagccacaaaaaaaactgataaacccagacaagaagataaagatgatctagatgtaacagagctcactaatgaagatcttttggatcagcttgtgaaatacggagtgaatcctggtcctattgtgggaacaaccaggaagctatatgagaaaaagcttttgaaactgagggaacaaggaacagaatcaagatcttctactcctctgccaacaatttcttcttcagcagaaaatacaaggcagaatggaagtaatgattctgacagatacagtgacaatgaagaaggaaagaagaaagaacacaagaaagtgaagtccactagggatattgttcctttttctgaacttggaactactccctctggtggtggattttttcagggtatttcttttcctgaaatctccacccgtcctcctttgggcagtaccgaactacaggcagctaagaaagtacatacttctaagggagacctacctagggagcctcttgttgccacaaacttgcctggcaggggacagttgcagaagttagcctctgaaaggaatttgtttatttcatgcaagtctagccatgataggtgtttagagaaaagttcttcgtcatcttctcagcctgaacacagtgccatgttggtctctactgcagcttctccttcactgattaaagaaaccaccactggttactataaagacatagtagaaaatatttgcggtagagagaaaagtggaattcaaccattatgtcctgagaggtcccatatttcagatcaatcgcctctctccagtaaaaggaaagcactagaagagtctgagagctcacaactaatttctccgccacttgcccaggcaatcagagattatgtcaattctctgttggtccagggtggggtaggtagtttgcctggaacttctaactctatgcccccactggatgtagaaaacatacagaagagaattgatcagtctaagtttcaagaaactgaattcctgtctcctccaagaaaagtccctagactgagtgagaagtcagtggaggaaagggattcaggttcctttgtggcatttcagaacatacctggatccgaactgatgtcttcttttgccaaaactgttgtctctcattcactcactaccttaggtctagaagtggctaagcaatcacagcatgataaaatagatgcctcagaactatcttttcccttccatgaatctattttaaaagtaattgaagaagaatggcagcaagttgacaggcagctgccttcactggcatgcaaatatccagtttcttccagggaggcaacacagatattatcagttccaaaagtagatgatgaaatcctagggtttatttctgaagccactccactaggaggtattcaagcagcctccactgagtcttgcaatcagcagttggacttagcactctgtagagcatatgaagctgcagcatcagcattgcagattgcaactcacactgcctttgtagctaaggctatgcaggcagacattagtcaagctgcacagattcttagctcagatcctagtcgtacccaccaagcgcttgggattctgagcaaaacatatgatgcagcctcatatatttgtgaagctgcatttgatgaagtgaagatggctgcccataccatgggaaatgccactgtaggtcgtcgatacctctggctgaaggattgcaaaattaatttagcttctaagaataagctggcttccactccctttaaaggtggaacattatttggaggagaagtatgcaaagtaattaaaaagcgtggaaataaacactag

SEQ ID NO: 12 - protein, 694 aa, Homo sapiens
MPEFLEDPSVLTKDKLKSELVANNVTLPAGEQRKDVYVQLYLQHLTARNRPPLPAGTNSKGPPDFSSDEEREPTPVLGSGAAAAGRSRAAVGRKATKKTDKPRQEDKDDLDVTELTNEDLLDQLVKYGVNPGPIVGTTRKLYEKKLLKLREQGTESRSSTPLPTISSSAENTRQNGSNDSDRYSDNEEGKKKEHKKVKSTRDIVPFSELGTTPSGGGFFQGISFPEISTRPPLGSTELQAAKKVHTSKGDLPREPLVATNLPGRGQLQKLASERNLFISCKSSHDRCLEKSSSSSSQPEHSAMLVSTAASPSLIKETTTGYYKDIVENICGREKSGIQPLCPERSHISDQSPLSSKRKALEESESSQLISPPLAQAIRDYVNSLLVQGGVGSLPGTSNSMPPLDVENIQKRIDQSKFQETEFLSPPRKVPRLSEKSVEERDSGSFVAFQNIPGSELMSSFAKTVVSHSLTTLGLEVAKQSQHDKIDASELSFPFHESILKVIEEEWQQVDRQLPSLACKYPVSSREATQILSVPKVDDEILGFISEATPLGGIQAASTESCNQQLDLALCRAYEAAASALQIATHTAFVAKAMQADISQAAQILSSDPSRTHQALGILSKTYDAASYICEAAFDEVKMAAHTMGNATVGRRYLWLKDCKINLASKNKLASTPFKGGTLFGGEVCKVIKKRGNKH

SEQ ID NO: 13 - protein, 21 aa, Homo sapiens
  MISC_FEATURE: Partial sequence of IPF1, showing high score in the local alignment between IPF1 and HNF3G, No.1
PPGLSASPQPSSVAPRRPQEP

SEQ ID NO: 14 - protein, 8 aa, Homo sapiens
  MISC_FEATURE: Partial sequence of IPF1, showing high score in the local alignment between IPF1 and HNF3G, No.2
PEGAEPGV

SEQ ID NO: 15 - protein, 12 aa, Homo sapiens
  MISC_FEATURE: Partial sequence of IPF1, showing high score in the local alignment between IPF1 and HNF3G, No.3
PPPHPFPGALGA

SEQ ID NO: 16 - protein, 22 aa, Homo sapiens
  MISC_FEATURE: Partial sequence of HNF3G, showing high score in the local alignment between IPF1 and HNF3G, No.1
PGGLPASPLPSGPLAPPAPAAP

SEQ ID NO: 17 - protein, 8 aa, Homo sapiens
  MISC_FEATURE: Partial sequence of HNF3G, showing high score in the local alignment between IPF1 and HNF3G, No.2
AEGGEPGV

SEQ ID NO: 18 - protein, 12 aa, Homo sapiens
  MISC_FEATURE: Partial sequence of HNF3G, showing high score in the local alignment between IPF1 and HNF3G, No.3
PLSSPYPGGLPA

SEQ ID NO: 19 - protein, 23 aa, Homo sapiens
  MISC_FEATURE: Partial sequence of IPF1, showing high score in the local alignment between IPF1 and PHF1, No.1
HPFPGALGALEQGSPPDISPYEV

SEQ ID NO: 20 - protein, 9 aa, Homo sapiens
  MISC_FEATURE: Partial sequence of IPF1, showing high score in the local alignment between IPF1 and PHF1, No.2
AVTSGEELL

SEQ ID NO: 21 - protein, 23 aa, Homo sapiens
  MISC_FEATURE: Partial sequence of PHF1, showing high score in the local alignment between IPF1 and PHF1, No.1
HPSASTAGTSGDSGPPDRSPLEL

SEQ ID NO: 22 - protein, 9 aa, Homo sapiens
  MISC_FEATURE: Partial sequence of PHF1, showing high score in the local alignment between IPF1 and PHF1, No.2
AALPGEELL

SEQ ID NO: 23 - protein, 57 aa, Homo sapiens
  MISC_FEATURE: Partial sequence of IPF1, showing high score in the local alignment between IPF1 and DLX4, No.1
KRTRTAYTRAQLLELEKEFLFNKYISRPRRVELAVMLNLTERHIKIWFQNRRMKWKK

SEQ ID NO: 24 - protein, 15 aa, Homo sapiens
  MISC_FEATURE: Partial sequence of IPF1, showing high score in the local alignment between IPF1 and DLX4, No.2
SPQPSSVAPRRPQEP

SEQ ID NO: 25 - protein, 57 aa, Homo sapiens
  MISC_FEATURE: Partial sequence of DLX4, showing high score in the local alignment between IPF1 and DLX4, No.1
RKPRTIYSSLQLQHLNQRFQHTQYLALPERAQLAAQLGLTQTQVKIWFQNKRSKYKK

SEQ ID NO: 26 - protein, 12 aa, Homo sapiens
  MISC_FEATURE: Partial sequence of DLX4, showing high score in the local alignment between IPF1 and DLX4, No.2
SPEPSERRPQAP

SEQ ID NO: 27 - protein, 15 aa, Homo sapiens
  MISC_FEATURE: Partial sequence of IPF1, showing high score in the local alignment between IPF1 and TCF4, No.1
HHHLPAQLALPHPPA

SEQ ID NO: 28 - protein, 16 aa, Homo sapiens
  MISC_FEATURE: Partial sequence of IPF1, showing high score in the local alignment between IPF1 and TCF4, No.2
RLPPGLSASPQPSSVA

SEQ ID NO: 29 - protein, 26 aa, Homo sapiens
  MISC_FEATURE: Partial sequence of IPF1, showing high score in the local alignment between IPF1 and TCF4, No.3
GPAPEFSASPPACLYMGRQPPPPPPH

SEQ ID NO: 30 - protein, 15 aa, Homo sapiens
  MISC_FEATURE: Partial sequence of TCF4 showing high score in the local alignment between IPF1 and TCF4, No.1
HSLLPNQVPVPQLPV

SEQ ID NO: 31 - protein, 16 aa, Homo sapiens
  MISC_FEATURE: Partial sequence of TCF4 showing high score in the local alignment between IPF1 and TCF4, No.2
KVPPGLPSSVYAPSAS

SEQ ID NO: 32 - protein, 26 aa, Homo sapiens
  MISC_FEATURE: Partial sequence of TCF4 showing high score in the local alignment between IPF1 and TCF4, No.3
GSPPSLSAGTAVWSRNGGQASSSPNY

SEQ ID NO: 33 - protein, 14 aa, Homo sapiens
  MISC_FEATURE: Partial sequence of IPF1 showing high score in the local alignment between IPF1 and TMPO
FQRGPAPEFSASPP

SEQ ID NO: 34 - protein, 14 aa, Homo sapiens
  MISC_FEATURE: Partial sequence of TMPO showing high score in the local alignment between IPF1 and TMPO
FQGISFPEISTRPP

SEQ ID NO: 35 - DNA, 37 bp, Artificial Sequence
  Desined oligonucleotide based on sequence of SEQ ID NO 3 for use as a primer (Human HNF3G)
gctagcctgggctcagtgaagatggaggcccatgacc

SEQ ID NO: 36 - DNA, 37 bp, Artificial Sequence
  Desined oligonucleotide based on sequence of SEQ ID NO 3 for use as a reverse primer (Human HNF3G)
ctcgagctaggatgcattaagcaaagagcgggaatag

SEQ ID NO: 37 - DNA, 26 bp, Artificial Sequence
  Desined oligonucleotide based on sequence of SEQ_ID NO5 for use as a primer (Human PHF1)
gcgctagcgcgcagcccccccggctg

SEQ ID NO: 38 - DNA, 26 bp, Artificial Sequence
  Desined oligonucleotide based on sequence of SEQ_ID NO5 for use as a reverse primer (Human PHF1)
gcctcgagtcagaagatgccccctcc

SEQ ID NO: 39 - DNA, 29 bp, Artificial Sequence
  Desined oligonucleotide based on sequence of SEQ ID NO 7 for use as a primer (Human DLX4)
acctgcagagcaccctcaggaactcgagg

SEQ ID NO: 40 - DNA, 29 bp, Artificial Sequence
  Desined oligonucleotide based on sequence of SEQ ID NO 7 for use as a reverse primer (Human DLX4)
gacagtctcaactccagccaatggctctc

SEQ ID NO: 41 - DNA, 31 bp, Artificial Sequence
  Desined oligonucleotide based on mouse HNF 3G gene for use as a primer
tgcctgtagagagaccgaagcactcggttcc

SEQ ID NO: 42 - DNA, 30 bp, Artificial Sequence
  Desined oligonucleotide based on mouse HNF 3G gene for use as a reverse primer
agctaatgggtcatatcacagtaatagacc

SEQ ID NO: 43 - DNA, 30 bp, Artificial Sequence
  Desined oligonucleotide based on mouse PH F1 gene for use as a primer
gagactcatccaggtctcttgagtctgacc

SEQ ID NO: 44 - DNA, 30 bp, Artificial Sequence
  Desined oligonucleotide based on mouse PH F1 gene for use as a reverse primer
tccaggtacatcccagctgtaggtgaggtc

SEQ ID NO: 45 - DNA, 29 bp, Artificial Sequence
  Desined oligonucleotide based on mouse DL X4 gene for use as a primer
gctggcagaaagagtagacagcgtgcggg

SEQ ID NO: 46 - DNA, 27 bp, Artificial Sequence
  Desined oligonucleotide based on mouse DL X4 gene for use as a reverse primer
agcaggtgctcgggtcctgggcttgac

SEQ ID NO: 47 - protein, 11 aa, Homo sapiens
  MISC_FEATURE: Partial sequence of IPF1 showing high score in the local alignment between IPF1 and HNF3G, No.4
PPGLSASPQPS

SEQ ID NO: 48 - protein, 7 aa, Homo sapiens
  MISC_FEATURE: Partial sequence of IPF1 showing high score in the local alignment between IPF1 and HNF3G, No.5
EGAEPGV

SEQ ID NO: 49 - protein, 8 aa, Homo sapiens
  MISC_FEATURE: Partial sequence of IPF1 showing high score in the local alignment between IPF1 and HNF3G, No.6
PFPGALGA

SEQ ID NO: 50 - protein, 11 aa, Homo sapiens
  MISC_FEATURE: Partial sequence of HNF3G showing high score in the local alignment between IPF1 and HNF3G, No.4
PGGLPASPLPS

SEQ ID NO: 51 - protein, 7 aa, Homo sapiens
  MISC_FEATURE: Partial sequence of HNF3G showing high score in the local alignment between IPF1 and HNF3G, No.5
EGGEPGV

SEQ ID NO: 52 - protein, 8 aa, Homo sapiens
  MISC_FEATURE: Partial sequence of HNF3G showing high score in the local alignment between IPF1 and HNF3G, No.6
PYPGGLPA

SEQ ID NO: 53 - protein, 8 aa, Homo sapiens
  MISC_FEATURE: Partial sequence of IPF1 showing high score in the local alignment between IPF1 and PHF1, No.3
PPDISPYE

SEQ ID NO: 54 - protein, 5 aa, Homo sapiens
  MISC_FEATURE: Partial sequence of IPF1 showing high score in the local alignment between IPF1 and PHF1, No.4
GEELL

SEQ ID NO: 55 - protein, 8 aa, Homo sapiens
  MISC_FEATURE: Partial sequence of PHF1 showing high score in the local alignment between IPF1 and PHF1, No.3
PPDRSPLE

SEQ ID NO: 56 - protein, 5 aa, Homo sapiens
  MISC_FEATURE: Partial sequence of PHF1 showing high score in the local alignment between IPF1 and PHF1, No.4
GEELL

SEQ ID NO: 57 - protein, 14 aa, Homo sapiens
  MISC_FEATURE: Partial sequence of IPF1 showing high score in the local alignment between IPF1 and DLX4, No.3
IKIWFQNRRMKWKK

SEQ ID NO: 58 - protein, 5 aa, Homo sapiens
  MISC_FEATURE: Partial sequence of IPF1 showing high score in the local alignment between IPF1 and DLX4, No.4
SPQPS

SEQ ID NO: 59 - protein, 6 aa, Homo sapiens
  MISC_FEATURE: Partial sequence of IPF1 showing high score in the local alignment between IPF1 and DLX4, No.5
RRPQEP

SEQ ID NO: 60 - protein, 14 aa, Homo sapiens
  MISC_FEATURE: Partial sequence of DLX4 showing high score in the local alignment between IPF1 and DLX4, No.3
VKIWFQNKRSKYKK

SEQ ID NO: 61 - protein, 5 aa, Homo sapiens
  MISC_FEATURE: Partial sequence of DLX4 showing high score in the local alignment between IPF1 and DLX4, No.4
SPEPS

SEQ ID NO: 62 - protein, 6 aa, Homo sapiens
  MISC_FEATURE: Partial sequence of DLX4 showing high score in the local alignment between IPF1 and DLX4, No.5
RRPQAP

SEQ ID NO: 63 - protein, 7 aa, Homo sapiens
  MISC_FEATURE: Partial sequence of IPF1 showing high score in the local alignment between IPF1 and TCF4, No.4
HHHLPAQ

SEQ ID NO: 64 - protein, 7 aa, Homo sapiens
  MISC_FEATURE: Partial sequence of IPF1 showing high score in the local alignment between IPF1 and TCF4, No.5
PPGLSAS

SEQ ID NO: 65 - protein, 8 aa, Homo sapiens
  MISC_FEATURE: Partial sequence of IPF1 showing high score in the local alignment between IPF1 and TCF4, No.6
GPAPEFSA

SEQ ID NO: 66 - protein, 7 aa, Homo sapiens
  MISC_FEATURE: Partial sequence of TCF4 showing high score in the local alignment between IPF1 and TCF4, No.4
HSLLPNQ

SEQ ID NO: 67 - protein, 7 aa, Homo sapiens
  MISC_FEATURE: Partial sequence of TCF4 showing high score in the local alignment between IPF1 and TCF4, No.5
PPGLPSS

SEQ ID NO: 68 - protein, 8 aa, Homo sapiens
  MISC_FEATURE: Partial sequence of TCF4 showing high score in the local alignment between IPF1 and TCF4, No.6
GSPPSLSA

SEQ ID NO: 69 - protein, 14 aa, Homo sapiens
  MISC_FEATURE: Partial sequence of IPF1 showing high score in the local alignment between IPF1 and TMPO, No.2
FQRGPAPEFSASPP

SEQ ID NO: 70 - protein, 14 aa, Homo sapiens
  MISC_FEATURE: Partial sequence of TMPO showing high score in the local alignment between IPF1 and TMPO, No.2
FQGISFPEISTRPP

(19) United States
(12) Patent Application Publication
(10) Pub. No.: US 2006/0276387 A1
(43) Pub. Date: Dec. 7, 2006

(54) Method of controlling transcription insulin gene

(76) Inventors: Hirofumi Doi, Chiba (JP); Kensaku Imai, Chiba (JP); Naoya Wada, Chiba (JP)

Correspondence Address: GREENBLUM & BERNSTEIN, P.L.C., 1950 ROLAND CLARKE PLACE, RESTON, VA, 20191, US

(21) Appl. No.: 10/544,061

(22) PCT Filed: Nov. 26, 2004
(86) PCT No.: PCT/JP04/18068

§ 371 Date: Mar. 20, 2006

(30) Foreign Application Priority Data
Nov. 28, 2003 (JP) 2003398529

(51) Int. Cl. A61K 38/22; C12Q 1/68; G01N 33/53

(52) U.S. Cl. 514/12; 435/6; 435/7.1

(57) ABSTRACT

A method for promoting insulin gene transcription, which comprises the step of inhibiting binding of IPF1 and any one of proteins selected from the following group: (i) HNF3G, (ii) PHF1, and (iii) DLX4; and a method for screening a substance that promotes insulin gene transcription, which comprises the step of bringing a test substance into contact with IPF1 and/or any one of proteins selected from the following group under a condition that allows the binding of IPF1 and said protein and then determining whether or not the test substance inhibits the binding of IPF1 and said protein by detecting presence or absence, or change of a signal and/or a marker generated by the binding of IPF1 and said protein in a system in which the signal and/or the marker can be detected: (i) HNF3G, (ii) PHF1, and (iii) DLX4.

### TECHNICAL FIELD

The present invention relates to a method for regulating insulin gene transcription. More specifically, the present invention relates to a method for regulating insulin gene transcription, which comprises the step of inhibiting binding of insulin promoter factor 1 (hereinafter, abbreviated as “IPF1” in the specification) and a protein that binds to IPF1.

### BACKGROUND ART

IPF1, a transcription factor expressed in β-cells of the pancreas, is a factor for promoting expressions of genes important for glycometabolism such as insulin, glucokinase, and GLUT2 (as reviews, see, Diabetologia, 44, 1203-1214, 2001; Eur. J. Endocrinol., 146, 129-141, 2002; Diabetologia, 45, 309-326, 2002). Deficiency of IPF1 function causes abnormal glycometabolism and results in onset of hereditary type 2 diabetes, i.e., maturity-onset diabetes of the young (MODY4, J. Clin. Invest., 104, R41-R48, 1999). Therefore, IPF1 is considered to be an important factor for glycometabolic system and functional maintenance of the pancreas such as insulin secretion.

It has been reported that IPF1 is phosphorylated via the signal transduction systems of phosphatidylinositol 3-kinase and stress-activated protein kinase, which is triggered by glucose stimulation, and then translocated into the nucleus to accelerate insulin gene promoter activity (J. Biol. Chem. 272, 20936-20944, 1997; J. Biol. Chem. 274, 1011-1016, 1999). However, any enzyme that catalyzes the phosphorylation of IPF1 has not yet been identified so far. Further, it has been reported that IPF1-dependent insulin gene promoter activity is inhibited by hepatocyte nuclear factor-1α (HNF-1α, Endocr. Res. 27, 63-74, 2001), and this inhibition is considered to be caused by competitive inhibition by HNF-1 a against the binding of IPF1 to the insulin gene promoter region. However, it has not yet been clarified so far whether or not IPF1 and HNF-1α directly bind to each other.

Several factors regulating gene transcription are known. For example, hepatocyte nuclear factor 3-gamma (HNF3G) is a transcription factor having a forkhead box and is considered to be a transcription-regulating factor of liver-specific genes (Genomics, 20, 377-385, 1994; Genomics, 39, 417-419, 1997; Mol. Cell. Biol., 18, 4245-4251, 1998). Distal-less homeobox 4 (DLX4, also sometimes called as BP1) is a transcription factor containing a homeodomain and is known to inhibit transcription of β-globin gene (Mol. Cell. Biol., 22, 2505-2514, 2002). Transcription factor 4 (TCF4) is a transcription factor having a helix-loop-helix structure and is known to bind to an initiator element of somatostatin receptor II gene or an enhancer elements of immunoglobulin genes to activate transcription (EMBO J., 15, 6680-6690; Science, 247, 467-470). Further, PHD finger protein 1 (PHF1) is a protein having a PHD finger domain and is speculated to be a transcription-regulating factor, although functions thereof remain unknown (Genomics, 48, 381-383, 1998). However, it has never been known so far that these transcription factors are factors for regulating insulin gene transcription. Furthermore, thymopoietin (TMPO), a class of nucleoprotein, is considered to possibly participate in maintenance of a nuclear structure and cell cycle. However, details of functions thereof remained unclarified (Genomics, 28, 198-205, 1995; Genome Res., 6, 361-370, 1996). It has not been reported that this protein is involved in insulin gene transcription.

### DISCLOSURE OF THE INVENTION

As explained above, IPF1 is an important transcription promoting factor in insulin gene transcription. Accordingly, identification of a protein that binds to IPF1 is extremely important for providing a means for prophylactic and/or therapeutic treatment of diabetes. Further, a means for regulating insulin gene transcription may possibly be provided on the basis of inhibition of the binding of IPF1 and the protein. Therefore, an object of the present invention is to provide a protein that binds to IPF1, and further provide a means for regulating insulin gene transcription by inhibiting the binding of IPF1 and the protein.

In order to identify a protein that binds to IPF1 and elucidate functions thereof, the inventors of the present invention divided the amino acid sequence of IPF1 into oligopeptides each having given lengths, and searched databases for proteins that have the amino acid sequences of those oligopeptides or amino acid sequences homologous to the amino acid sequences, and then according to the prediction method described in International Patent Publication WO01/67299, they performed local alignments of the selected proteins and IPF1 and predicted that proteins giving a high local alignment score are proteins that successfully bind to IPF1. As a result, they found several types of proteins which contains oligopeptides having homology to the oligopeptides consisting of IPF1-derived amino acid sequences. Further, the inventors of the present invention found that these proteins successfully bind to IPF1, and found a method for regulating insulin gene transcription on the basis of the binding. They also found that these proteins have inhibitory actions on insulin promoter activity. The present invention was achieved on the basis of the above findings.

The present invention thus provides a method for promoting insulin gene transcription, which comprises the step of inhibiting binding of IPF1 and any one of protein selected from the following group:

(i) hepatocyte nuclear factor 3-gamma (HNF3G),

(ii) PHD finger protein 1 (PHF1), and

(iii) distal-less homeobox 4 (DLX4).

Further, the present invention also provides a method for screening a substance that inhibits binding of IPF1 and any one of protein selected from the following group, which comprises the step of bringing a test substance into contact with IPF1 and/or the protein under a condition that allows binding of IPF1 and said protein and then determining whether or not the test substance inhibits the binding of IPF1 and said protein by detecting the presence or absence, or change of a signal and/or marker generated by binding of IPF1 and said protein in a system in which the signal and/or the marker can be detected:

(i) HNF3G,

(ii) PHF1,

(iii) DLX4,

(iv) transcription factor4 (TCF4), and

(v) thymopoietin (TMPO).

Further, the present invention also provides a method for screening a substance that promotes insulin gene transcription, which comprises the step of bringing a test substance into contact with IPF1 and/or any one of protein selected from the following group under a condition that allows binding of IPF1 and said protein and then determining whether or not the test substance inhibits the binding of IPF1 and said protein by detecting the presence or absence, or change of a signal and/or marker generated by binding of IPF1 and said protein in a system in which the signal and/or the marker can be detected:

(i) HNF3G,

(ii) PHF1, and

(iii) DLX4.

The present invention also provides a method for screening a substance that promotes insulin gene transcription, which comprises the step of bringing a test substance into contact with IPF1 and/or any one of protein selected from the following group under a condition that allows binding of IPF1 and said protein to determine whether or not insulin gene transcription is promoted:

(i) HNF3G,

(ii) PHF1, and

(iii) DLX4.

From another aspect, the present invention provides a substance screened by any of the above screening methods.

The present invention also provides a medicament for prophylactic treatment and/or therapeutic treatment of a disease caused by a reduced amount of gene product of the insulin gene, which medicament inhibits binding of IPF1 and any one of protein selected from the following group:

(i) HNF3G,

(ii) PHF1, and

(iii) DLX4; and

a medicament for prophylactic treatment and/or therapeutic treatment of diabetes, which inhibits binding of IPF1 and any one of protein selected from the following group:

(i) HNF3G,

(ii) PHF1, and

(iii) DLX4.

According to a preferred embodiment of these medicaments, the present invention provides a medicament containing, as an active ingredient, a substance screened by any of the aforementioned screening methods.

The present invention further provides a method for prophylactic treatment and/or therapeutic treatment of a disease caused by a reduced amount of gene product of the insulin gene, which comprises the step of inhibiting binding of IPF1 and any one of protein selected from the following group:

(i) HNF3G,

(ii) PHF1, and

(iii) DLX4; and

a method for prophylactic treatment and/or therapeutic treatment of diabetes, which comprises the step of inhibiting binding of IPF1 and any one of protein selected from the following group:

(i) HNF3G,

(ii) PHF1, and

(iii) DLX4.

According to a preferred embodiment of these methods, the present invention provides a method comprising the step of administering a substance screened by any of the aforementioned screening methods.

From a further aspect, the present invention provides a kit of reagents used for any of the aforementioned screening methods, which comprises:

(a) IPF1 and/or a DNA encoding IPF1, and

(b) a protein binding to IPF1 and/or a DNA encoding said protein. According to a preferred embodiment, the present invention provides a kit of reagents, which comprises:

(a) IPF1 and/or a DNA encoding IPF1, and

(b) one or more kinds of proteins selected from the following group and/or DNAs encoding said proteins:

(i) HNF3G,

(ii) PHF1, and

(iii) DLX4.

### BRIEF DESCRIPTION OF THE DRAWINGS

FIG. 1 shows results of local alignments of oligopeptides PPGLSASPQPS, EGAEPGV and PFPGALGA consisting of IPF1-derived amino acid residues with PGGLPASPLPS, EGGEPGV and PYPGGLPA which are homologous oligopeptides in HNF3G.

FIG. 2 shows results of local alignments of oligopeptides PPDISPYE and GEELL consisting of IPF1-derived amino acid residues with PPDRSPLE and GEELL which are homologous oligopeptides in PHF1.

FIG. 3 shows results of local alignments of oligopeptides IKIWFQNRRMKWKK, SPQPS and RRPQEP consisting of IPF1-derived amino acid residues with VKIWFQNKRSKYKK, SPEPS and RRPQAP which are homologous oligopeptides in DLX4.

FIG. 4 shows results of local alignments of oligopeptides HHHLPAQ, PPGLSAS and GPAPEFSA consisting of IPF1-derived amino acid residues with HSLLPNQ, PPGLPSS and GSPPSLSA which are homologous oligopeptides in TCF4.

FIG. 5 shows results of local alignment of an oligopeptide FQRGPAPEFSASPP consisting of IPF1-derived amino acid residues with FQGISFPEISTRPP which is a homologous oligopeptide in TMPO.

FIG. 6 shows results of a binding test of IPF1 to HNF3G, PHF1, DLX4, TCF4 or TMPO.

FIG. 7 shows results of detection of IPF1-dependent human insulin promoter activity in a HeLa cell system.

FIG. 8 shows effect of overexpression of HNF3G, PHF1 and DLX4 on IPF1-dependent human insulin promoter activity in a HeLa cell system.

FIG. 9 shows effect of overexpression of HNF3G, PHF1 and DLX4 on human insulin promoter activity in an MIN6 cell system.

FIG. 10 shows results of confirmation of expressions of human HNF3G, human PHF1 and human DLX4 in human pancreas (RT-PCR).

FIG. 11 shows results of confirmation of expressions of mouse HNF3G, mouse PHF1 and mouse DLX4 in MIN6 cells (RT-PCR).

### BEST MODE FOR CARRYING OUT THE INVENTION

The proteins (i) to (v) used in the present invention (also referred to as “wild type interactive proteins”) are proteins represented by the amino acid sequences of SEQ ID NOS: 4, 6, 8, 10 and 12 in the sequence listing, respectively. IPF1 used in the present invention (also referred to as “wild type IPF1”) is a protein represented by the amino acid sequence of SEQ ID NO: 2 in the sequence listing. Those skilled in the art can easily obtain the wild type interactive proteins and wild type IPF1 in view of these descriptions. For example, the proteins can be recovered and purified from samples in which production of these proteins are observed (for example, cells derived from the human pancreas) by a purification method known per se (affinity chromatography by using a monoclonal antibody recognizing each protein as an antigen).

Further, in the present specification, the proteins (i) to (v) encompass proteins which have amino acid sequences including substitution, insertion or deletion of one to several amino acid in the amino acid sequence of the aforementioned wild type interactive proteins and have substantially the same insulin gene transcription regulating action as that of the aforementioned wild type interactive proteins, or having substantially the same insulin promoter activity inhibitory action as that of the aforementioned wild type interactive proteins in mammals in vivo including a human (these proteins are also referred to as “mutant type interactive proteins”).

Further, in the specification, IPF1 encompasses proteins which have amino acid sequences including substitution, insertion or deletion of one to several amino acids in the amino acid sequence of the aforementioned wild type IPF1 and have substantially the same insulin promoter activation action as that of the aforementioned wild type IPF1 or have substantially the same insulin gene transcription promoting action as that of the aforementioned wild type IPF1 in mammals in vivo including a human (these proteins are also referred to as “mutant type IPF1”).

In general, the mutant type interactive proteins preferably have an amino acid sequence having homology higher than a given level to the amino acid sequences of the wild type interactive proteins (for example, 70% or higher, preferably 80% or higher, more preferably 85% or higher, further preferably 90% or higher, and most preferably 95% or higher). Similarly, the mutant IPF1 preferably has an amino acid sequence having homology higher than a given level to the amino acid sequence of the wild type IPF1 (for example, 70% or higher, preferably 80% or higher, more preferably 85% or higher, further preferably 90% or higher, and most preferably 95% or higher).

Methods for obtaining genes encoding these mutant proteins are known. For example, the genes can be appropriately obtained by the methods described in Molecular Cloning: A Laboratory Manual (Ed. by Sambrook et al., Cold Spring Harbor Laboratory Press, Cold Spring Harbor, N.Y., 1989) and the like or similar methods thereto, and desired mutant proteins can be easily obtained. Whether or not a mutant protein obtained has substantially the same insulin gene transcription regulating action substantially as that of the wild type interactive proteins can be easily examined by those skilled in the art by using the method for detecting inhibitory action on insulin promoter activity specifically described in Example 3 of the present specification. Further, whether or not a mutant protein obtained has the same insulin promoter activation action as that of the wild type IPF1 can be easily examined by those skilled in the art by using the method for determining insulin promoter activity specifically described in Example 3 of the present specification.

The insulin gene transcription promotion method provided by the present invention is characterized to comprise the step of inhibiting binding of IPF1 with any one of protein selected from the group consisting of the aforementioned proteins (i), (ii) and (iii). Although it is not intended to be bound by any specific theory, the aforementioned proteins (i), (ii) and (iii) that bind to IPF1 are generally expected to inhibit the insulin promoter activity of IPF1. Therefore, a substance inhibiting said binding can activate the insulin promoter and thereby promote the insulin gene transcription by inhibiting the insulin promoter activity suppressing action of the aforementioned protein. The type of the aforementioned substance inhibiting said binding is not particularly limited, and examples thereof include low molecular compounds such as organic compounds, inorganic compounds, and sugar compounds, as well as high molecular compounds such as proteins including antibodies, nucleic acids including antisense nucleic acids, polysaccharides, and lipids. Further, the substance may be a natural or a non-natural substance.

In the specification, the binding of IPF1 with a certain protein means an interaction of IPF1 with the protein via a non-covalent bond such as hydrogen bond, hydrophobic bond, and electrostatic interaction so that IPF1 and the protein can form a complex. As for the “binding” herein referred to, the binding of IPF1 with said protein as a whole is sufficient. For example, amino acids constituting IPF1 or the protein may include amino acids which are not involved in the binding of IPF1 and said protein.

The binding of IPF1 with said protein can be detected by methods known per se such as identification of coprecipitates by an immunoprecipitation technique, two-hybrid analysis, pull-down analysis, Western blotting, and fluorescence resonance energy transfer, or any combination of these methods.

A substance having the aforementioned inhibitory action can be screened by, for example, the following method provided by the present invention. This method is for screening a substance that inhibits the binding of IPF1 with any one of proteins selected from the group consisting of the aforementioned proteins (i) to (v), and the method is characterized to comprise the step of bringing a test substance into contact with IPF1 and/or the protein under a condition that allows the binding of IPF1 and the protein, and then detecting the presence or absence, or change of a signal and/or marker generated by the binding of IPF1 with the protein in a system in which the signal and/or the marker can be detected to determine whether or not the test substance inhibits the binding of IPF1 and the protein. According to a preferred embodiment of the aforementioned method, a substance that inhibits the binding of IPF1 with any one of proteins selected from the group consisting of the aforementioned proteins (i), (ii) and (iii) can be screened.

The condition that allows the binding of IPF1 with said protein in the aforementioned step means, for example, a condition wherein IPF1 and the protein are co-expressed in a cell. Such a condition can be satisfied by transfecting a suitable vector, which is incorporated with polynucleotides coding for IPF1 and the protein, into a cell by a conventional genetic engineering technique.

The signal generated by the binding of IPF1 and the protein in the aforementioned step means a signal that is generated by the binding of IPF1 with the protein and can be directly detected on the basis of a physical or chemical property of the signal, per se. The marker generated by the binding of IPF1 and the protein means a marker that is generated by binding of IPF1 with the protein and can be indirectly detected by using a physical or biological property of the marker, per se, as an index. Examples of the signaling substances include luciferase, radioactive isotopes and the like. Examples of the markers include reporter genes such as the chloramphenicol acetyltransferase gene or the like, and epitope tags for detection such as 6×His-tag. Methods for detecting these signals or markers are well known to those skilled in the art.

When a test substance is allowed to coexist with IPF1 and the protein under a condition that allows the binding of IPF1 with the protein in the aforementioned step, it can be judged that the test substance inhibits the binding of IPF1 with the protein if a signal and/or marker generated by the binding of IPF1 and the protein is reduced as compared with a step where the test substance is not allowed to coexist, or the signal and/or marker disappears.

The type of the test substance used in the screening method of the present invention is not particularly limited, and any compound can be used as the test substance. The test substance may be any of low molecular compounds such as organic compounds, inorganic compounds, and sugar compounds, as well as high molecular compounds such as proteins, nucleic acids, polysaccharides, and lipids. Further, the substance may be a natural or a non-natural substance. Examples of a library that is subjected to the screening include a low molecular compound library, phage display library, combinatorial library and the like. However, the libraries are not limited to these examples.

In the aforementioned method, the step of detecting the presence or absence, or change of a signal and/or marker generated by the binding of IPF1 with the protein in a system in which the signal and/or the marker can be detected is performed as a step for determining whether or not the insulin gene transcription is promoted. As a result, a substance that promotes the insulin gene transcription can be screened according to the aforementioned method.

The present invention further provides a kit for performing the aforementioned screening. The kit is characterized to comprise (a) IPF1 and/or a DNA encoding IPF1, and (b) a protein that interacts with IPF1 and/or a DNA encoding the protein. The elements of the kit may be provided as proteins, or they may be provided in the forms of genes that can express the proteins, preferably recombinant vectors containing the genes or the like.

A medicament containing a substance screened by the method of the present invention as an active ingredient can be administered to a mammal including a human as a medicament for prophylactic treatment and/or therapeutic treatment of a disease caused by a reduced amount of a gene product of the insulin gene. An example of the disease caused by the reduced amount of the gene product of the insulin gene includes diabetes (including complications of diabetes). The administration route of the medicament of the present invention is not particularly limited, and the medicament can be orally or parenterally administered. As the medicament of the present invention, a substance as the active ingredient, per se, may be used. However, a pharmaceutical composition containing a pharmacologically and pharmaceutically acceptable additive together with the substance as an active ingredient is preferably prepared and administered.

For example, the medicament of the present invention containing a protein as an active ingredient can be prepared by an ordinary method for preparing a protein preparation. The medicament of the present invention containing a nucleic acid as an active ingredient can also be prepared by a means available in this field. In the specification, the term “nucleic acid” encompasses DNA and RNA. The medicament of the present invention containing a nucleic acid can be used, for example, in the form of a recombinant vector containing the nucleic acid as an active ingredient. The aforementioned recombinant vector may be incorporated with various sequences required to express the gene or promote gene expression in a suitable order so that a gene product can be efficiently expressed in vivo from the nucleic acid as an active ingredient. When the medicament of the present invention contains an antisense nucleic acid, the nucleic acid may be either DNA or RNA, and a total length thereof is not particularly limited. The antisense nucleic acid may be, for example, an oligonucleotide of 10 nucleotides or more, preferably 15 nucleotides or more, so as to allow complementary binding. When an antibody, preferably a monoclonal antibody, which can bind to the aforementioned protein is used as an active ingredient of the medicament of the present invention, the antibody can be produced by an ordinary method, and a monoclonal antibody that can specifically bind to the aforementioned protein can also be produced by a method generally used by those skilled in the art.

Examples of the pharmacologically and pharmaceutically acceptable additives include excipients, disintegrating agents or disintegrating aids, binders, lubricants, coating agents, dyes, diluents, bases, dissolving agents or dissolving aids, isotonic agents, pH modifiers, stabilizers, propellants, adhesion agents and the like. Examples of pharmaceutical compositions suitable for oral administration include tablets, capsules, powders, subtilized granules, granules, solutions, syrups and the like. Examples of pharmaceutical compositions suitable for parenteral administration include injections, drip infusions, suppositories, inhalants, transdermal preparations, eye drops, ear drops, ointments, creams, patches and the like. Doses of the medicament of the present invention are not particularly limited, and can be suitably selected depending on various conditions such as the type of the substance as an active ingredient, the purpose of therapeutic or preventive treatment, the age and symptom of a patient, and the route of administration. In general, doses can be selected from the range of about 0.001 to 1000 mg per day for an adult.

The present invention also provides a method for prophylactic and/or therapeutic treatment of a disease caused by a reduced amount of a gene product of the insulin gene. The method is characterized by inhibiting the binding of IPF1 and a protein that binds to the IPF1 (preferably, HNF3G, PHF1 or DLX4). An example of the disease caused by the reduced amount of the gene product of the insulin gene includes diabetes. The method can be implemented by using the aforementioned medicament.

### EXAMPLES

The present invention will be explained more specifically with reference to the following examples. However, the scope of the present invention is not limited to these examples.

### Example 1

### Prediction of Proteins Interacting with IPF1

Proteins interacting with IPF1 were predicted according to the prediction method described in International Patent Publication WO01/67299. The amino acid sequence of IPF1 was divided into oligopeptides having suitable lengths. Proteins having amino acid sequences of these oligopeptides or amino acid sequences with homology to the amino acid sequences of the above oligopeptides were searched in databases, and local alignment was performed for each of the obtained proteins and IPF1. The proteins that gave a high local alignment score was predicted to interact with IPF1. The high local alignment score was defined as a score of 25.0 or higher as in the method described in International Patent Publication WO01/67299.

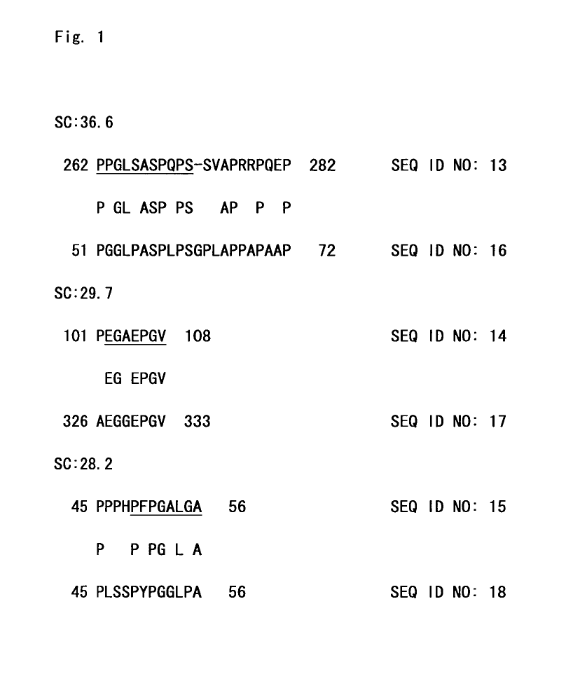

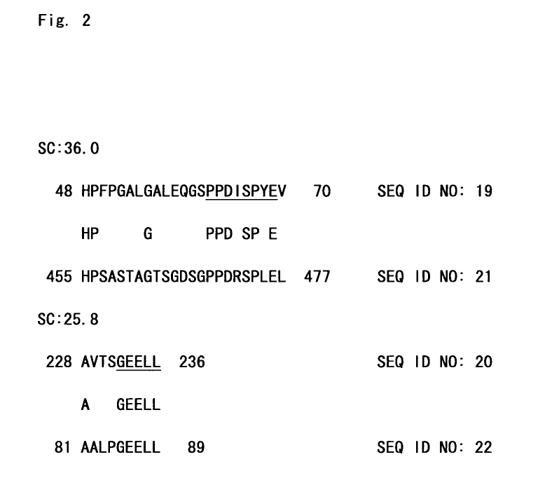

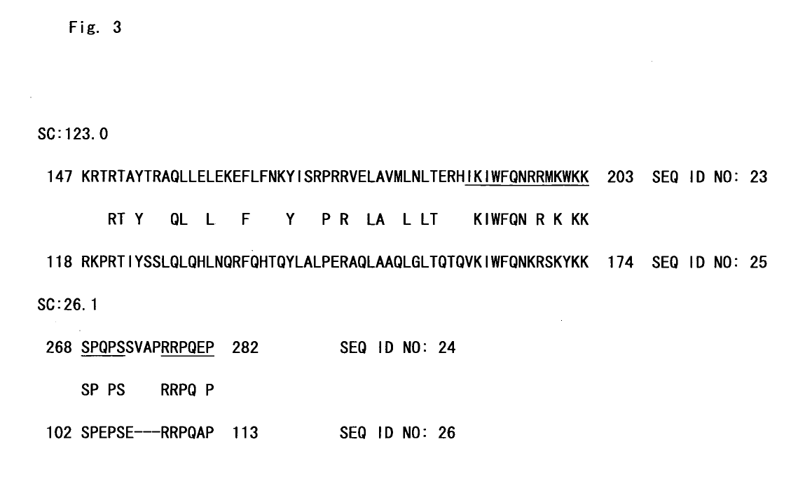

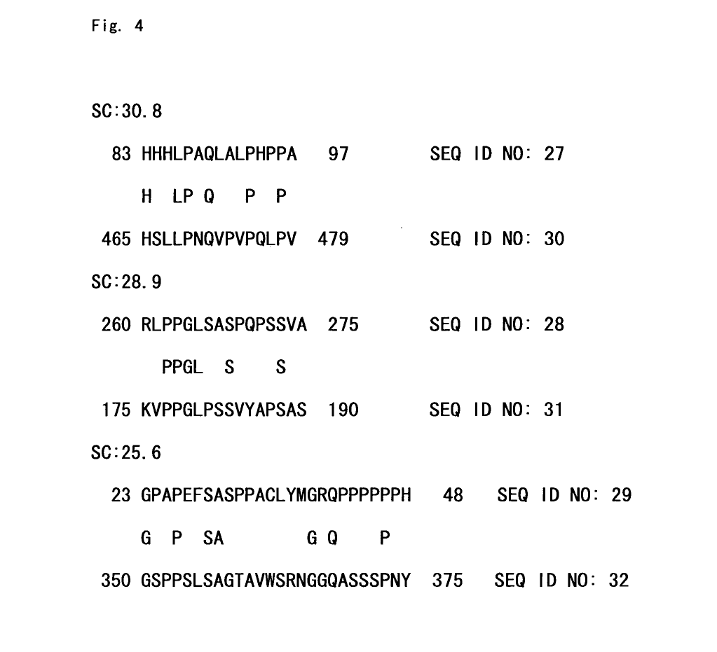

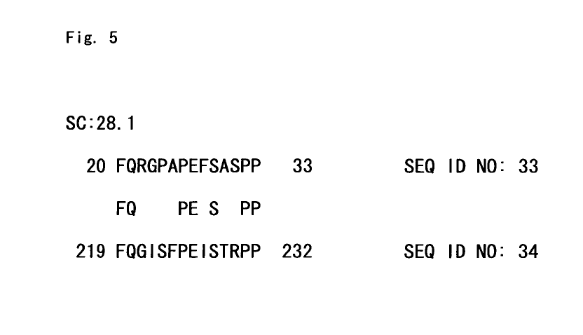

As a result of the prediction, the oligopeptides of SEQ ID NOS: 50, 51 and 52 having homology to the oligopeptides of SEQ ID NOS: 47, 48 and 49, each consisting of IPF1-derived amino acid residues, were found to exist in the amino acid sequence of HNF3G. The result of the local alignment of IPF1 and HNF3G is shown in FIG. 1. Further, the oligopeptides of SEQ ID NOS: 55 and 56 having homology to the oligopeptides of SEQ ID NOS: 53 and 54, each consisting of IPF1-derived amino acid residues, were found to exist in the amino acid sequence of PHF1 (FIG. 2), the oligopeptides of SEQ ID NOS: 60, 61 and 62 having homology to the oligopeptides of SEQ ID NOS: 57, 58 and 59, each consisting of IPF1-derived amino acid residues, were found to exist in the amino acid sequence of DLX4 (FIG. 3), the oligopeptides of SEQ ID NOS: 66, 67 and 68 having homology to the oligopeptides of SEQ ID NOS: 63, 64 and 65, each consisting of IPF1-derived amino acid residues, were found to exist in the amino acid sequence of TCF4 (FIG. 4), and the oligopeptide of SEQ ID NO: 70 having homology to the oligopeptide of SEQ ID NO: 69, consisting of IPF1-derived amino acid residues, were found to exist in the amino acid sequence of TMPO (FIG. 5).

### Example 2

### Binding Test to Human IPF1

Studies were made by applying the GST-pull down assay to examine whether or not human IPF1 binds to human HNF3G, human PHF1, human DLX4, human TCF4, or human TMPO.

<Materials>

(1) Cloning of Each cDNA

Human IPF1 cDNA was cloned from human liver-derived cDNA (Clontech), human HNF3G cDNA was cloned from human liver-derived cDNA (Clontech), human PHF1 cDNA, human TCF4 cDNA, and human TMPO cDNA were cloned from human brain-derived cDNA (Clontech), and human DLX4 cDNA was cloned from human placenta-derived cDNA (Clontech) by PCR.

(2) Various Expression Plasmids

Human IPF1 cDNA was introduced into pGEX-4T (Amersham Biosciences), a GST fusion protein expression vector, to construct pGEX-4T/IPF1 as an N-terminus GST-fused IPF1 expression plasmid. Further, each cDNA of human HNF3G, human PHF1, human DLX4, human TCF4, and human TMPO was introduced into pcDNA3.1(+) (Invitrogen), as being a vector for in vitro protein synthesis and expression in animal cells, to construct expression plasmids (pcDNA-HA-HNF3G, pcDNA-HA-PHF1, pcDNA-HA-DLX4, pcDNA-HA-TCF4, and pcDNA-HA-TMPO). In this construction, an HA tag-coding sequence was inserted at the 5′ end of each cDNA so as to have each protein expressed as an N-terminus HA-tagged protein. As a LacZ expression plasmid as a negative control, pCruzHA-LacZ (N-terminus HA-tagged LacZ expression plasmid, Santa Cruz) was used.

(3) Purification of N-Terminus GST-Fused IPF1 (GST-IPF1)

GST-IPF1 was expressed in Escherichia coli containing pGEX-4T/IPF1 and then purified by using Glutathione Sepharose 4B (Amersham Biosciences).

<Method>

(1) Binding Test by GST-Pull Down Assay

Human HNF3G, human PHF1, human DLX4, human TCF4, human TMPO, and LacZ were synthesized in vitro as ³⁵S-methionine-labeled proteins by using TNT Quick Coupled Transcription/Translation Systems (Promega). In a volume of 20 μl of a reaction mixture for synthesis and 5 μg of GST-IPF1 or GST were left standing in 500 μl of a binding buffer (40 mM HEPES, pH 7.5/50 mM KCl/5 mM MgCl₂/0.2 mM EDTA/1 mM DTT/0.5% NP-40) on ice for 1 hour. Then, the mixture was added with 20 μl (bed volume) of Glutathione Sepharose 4B and mixed gently overnight at 4° C. on a rocker, and then the beads were recovered by centrifugation. The beads were washed 4 times with 500 μl of the binding buffer, added with 20 μl of 2×SDS sample buffer (125 mM Tris-HCl, pH 6.8/4% SDS/20% glycerol/0.01% bromophenol blue) and boiled for 3 minutes, and then the supernatant was separated by 5-20% SDS-PAGE. Then, the binding proteins were detected by using BAS2000 (Fuji Photo Film).

<Results>

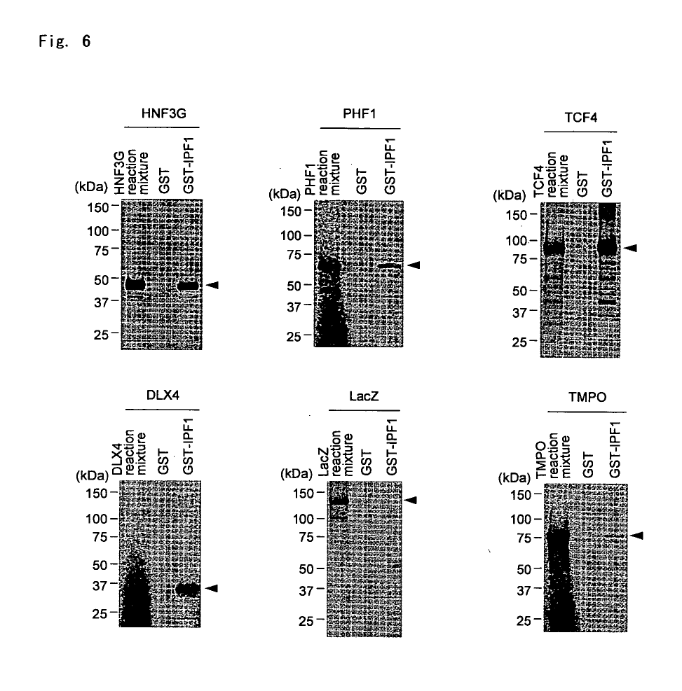

The bindings of IPF1 with HNF3G, PHF1, DLX4, TCF4, and TMPO were observed (FIG. 6). Whilst the binding of LacZ and IPF1 was not observed, which means that the detected binding of IPF1 with each protein was not non-specific (FIG. 6). In FIG. 6, GST (lane of “GST”) or GST-IPF1 (lane of “GST-IPF1”) was mixed with each protein synthesized as ³²S-methionine-labeled protein (HNF3G, PHF1, DLX4, TCF4, TMPO, and LacZ) (lanes of “input”) in an in vitro transcription/translation system, and GST or GST-IPF1 was recovered by using Glutathione Sepharose and separated by SDS-PAGE. Then, the bound proteins were detected by autoradiography. Each arrowhead points at the position of each protein (HNF3G, PHF1, DLX4, TCF4, TMPO, and LacZ) synthesized in the in vitro system.

### Example 3

### Inhibition of Human Insulin Gene Promoter Activity by Human HNF3G, Human PHF1, and Human DLX4

Among human HNF3G, human PHF1, human DLX4, human TCF4, and human TMPO which were found to bind to IPF1, human HNF3G, human PHF1, and human DLX4 were used to examine inhibitory action against human insulin gene promoter activity by using a reporter assay system.

<Materials>

(1) Cloning of Human Insulin Gene Promoter Region and Construction of Luciferase Reporter Plasmid

The human insulin gene promoter region (−392/+237, transcription initiation point being defined “+1”) was cloned from Human genomic DNA (Clontech) by PCR. The cloned promoter region was introduced into pGL3-Basic (Promega) as a luciferase reporter vector to construct pInsPro(−392/+237)-GL3 as a reporter plasmid for determining human insulin gene promoter activity.

(2) Various Expression Plasmids

Human IPF1 cDNA was introduced into pcDNA3.1(+) (Invitrogen) to construct an IPF1 expression plasmid (pcDNA-FLAG-IPF1). In this construction, a FLAG tag coding sequence was inserted into the 5′ end of IPF1 cDNA so as to have the protein expressed as an N-terminus FLAG-tagged protein. As expression plasmids for human HNF3G, human PHF1, and human DLX4, pcDNA-HA-HNF3G, pcDNA-HA-PHF1 and pcDNA-HA-DLX4 were used, respectively.

<Methods>

(1) Transfection and Reporter Assay

2×10⁵ HeLa cells were inoculated on a 6-well plate on the previous day and the cells were cultured overnight, and then transfection was performed by using FuGENE6 (Roche Diagnostics). As plasmids, 200 ng of pInsPro(−392/+237)-GL3, 400 ng of pcDNA-FLAG-IPF1, and 1 μg each of the expression plasmids (pcDNA-HA-HNF3G, pcDNA-HA-PHF1, and pcDNA-HA-DLX4) as well as 0.5 ng of pRL-SV40 (Promega) as an internal control were used. The total amount of DNA was adjusted with pcDNA3.1(+) (Invitrogen) to 1.6 μg. The cells were cultured for 48 hours, and luciferase activity was determined by using Dual-Luciferase Reporter Assay System (Promega). The measured values were corrected on the basis of the Renilla luciferase activity. When MIN6 cells, cells of mouse insulinoma-derived β-cell strain, were used, 5×10⁵ MIN6 cells were inoculated on a 6-well plate on the previous day and cultured overnight, and transfection was performed by using FuGENE6 (Roche Diagnostics). As plasmids, 200 ng of pInsPro(−392/+237)-GL3 and 1 μg each of the expression plasmids (pcDNA-HA-HNF3G, pcDNA-HA-PHF1, and pcDNA-HA-DLX4) as well as 5 ng of pRL-SV40 as an internal control were used. The total amount of DNA was adjusted with pcDNA3.1(+) (Invitrogen) to 1.2 μg. The cells were cultured for 48 hours, and then the luciferase activity was determined by the same method as described above.

<Results>

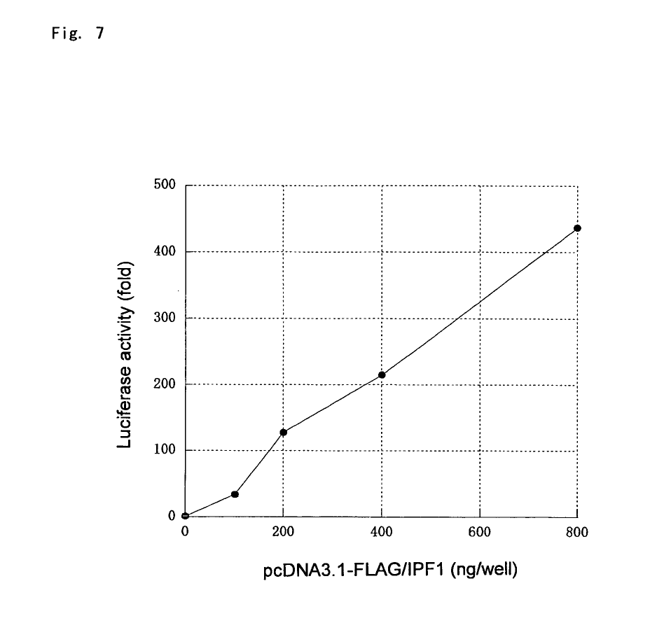

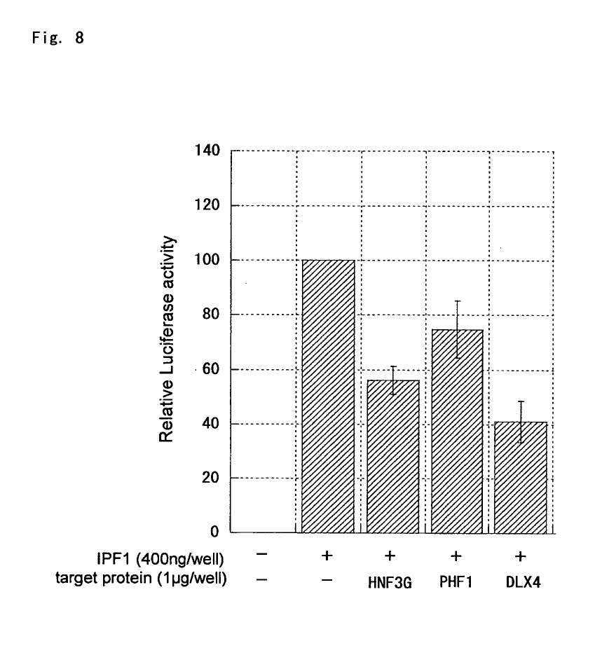

A system for detecting IPF1-dependent human insulin promoter activity was first constructed in which a luciferase reporter system in HeLa cells not expressing IPF1 was used. As a result, increase in human insulin promoter activity dependent on the amount of IPF1 expression plasmid was observed as shown in FIG. 7. In FIG. 7, the vertical axis represents relative luciferase activity based on the luciferase activity without pcDNA-FLAG-IPF1, which is taken as 1, and the horizontal axis represents the amount of introduced pcDNA-FLAG-IPF1. Then, effect of each protein was examined by using this system. As a result, it was revealed that IPF1-dependent human insulin promoter activity was inhibited by about 45%, about 25%, and about 60% due to overexpression of HNF3G, PHF1 and DLX4, respectively (FIG. 8). In FIG. 8, the vertical axis represents relative luciferase activity based on the luciferase activity obtained with introduction of pcDNA-FLAG-IPF1 alone, which was taken as 100. The symbol “−” means that any expression plasmid was not introduced, and “+” means that an expression plasmid was introduced. Further, HNF3G, PHF1, and DLX4 mean that each corresponding expression plasmid was introduced.

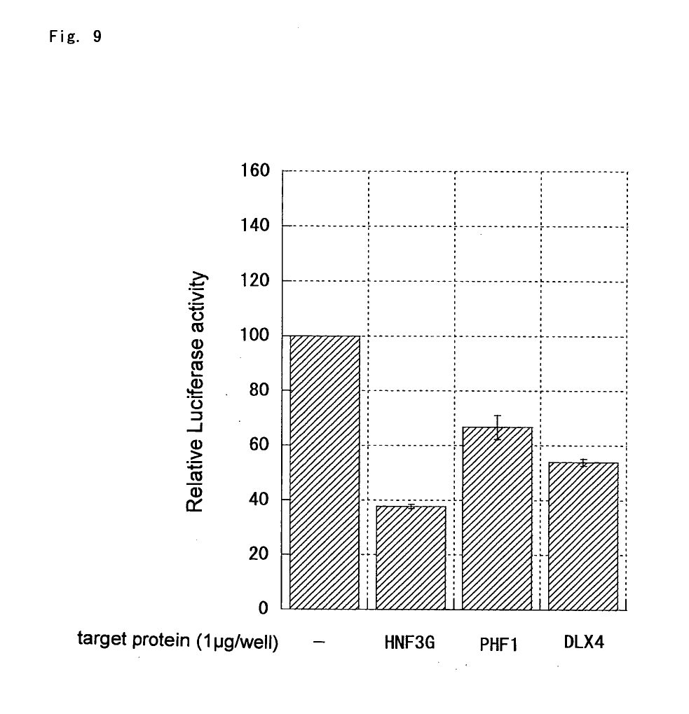

Then, effect of HNF3G, PHF1, and DLX4 on the human insulin promoter activity was examined by a reporter assay using MIN6 cells which are cells of a mouse insulinoma-derived β-cell strain intrinsically expressing IPF1. As a result, it was revealed that human insulin promoter activity was inhibited by about 60%, about 30%, and about 45% due to overexpression of HNF3G, PHF1, and DLX4, respectively (FIG. 9). In FIG. 9, the vertical axis represents relative luciferase activity based on the luciferase activity obtained without introduction of each expression plasmid, which was taken as 100. The symbol “−” means that any expression plasmid was not introduced, and “HNF3G,” “PHF1,” and “DLX4” mean that each corresponding expression plasmid was introduced. The above results revealed that HNF3G, PHF1, and DLX4 inhibited both of the IPF1-dependent human insulin gene promoter activity in the HeLa cell system and human insulin promoter activity in the MIN6 cell system.

### Example 4

### Confirmation of expression of HNF3G, PHF1, and DLX4 in human pancreas and MIN6 cells (RT-PCR)

Studies were made by applying the RT-PCR method to examine whether or not HNF3G, PHF1, and DLX4 were expressed in the human pancreas as the insulin secreting tissue, and in MIN6 cells as cells of a mouse insulinoma derived β-cell strain.

<Method>

(1) Confirmation of each mRNA by RT-PCR

cDNAs derived from the human pancreas, human brain, and human placenta were purchased from Invitrogen. As for the mouse MIN6 cells, total RNAs were prepared by using RNeasy Mini Kit (Qiagen) and then used to synthesize cDNAs derived from the MIN6 cells by using RNA PCR Kit (AMV) Ver.2.1 (Takara). PCR was performed by using each cDNA as a template, KOD Plus DNA Polymerase (Toyobo), and primers specific to each gene. Each reaction mixture was subjected to electrophoresis in a 2% agarose gel and stained with ethidium bromide to detect target PCR products.

The primers used were:

for human HNF3G,

oligonucleotide consisting of the nucleotide sequence of SEQ ID NO: 35, and oligonucleotide consisting of the nucleotide sequence of SEQ ID NO: 36, for human PHF1,

oligonucleotide consisting of the nucleotide sequence of SEQ ID NO: 37, and oligonucleotide consisting of the nucleotide sequence of SEQ ID NO: 38, and for human DLX4.,

oligonucleotide consisting of the nucleotide sequence of SEQ ID NO: 39, and oligonucleotide consisting of the nucleotide sequence of SEQ ID NO: 40.

Further used are:

for mouse HNF3G,

oligonucleotide consisting of the nucleotide sequence of SEQ ID NO: 41, and oligonucleotide consisting of the nucleotide sequence of SEQ ID NO: 42, for mouse PHF1,

oligonucleotide consisting of the nucleotide sequence of SEQ ID NO: 43, and oligonucleotide consisting of the nucleotide sequence of SEQ ID NO: 44, and for mouse DLX4,

oligonucleotide consisting of the nucleotide sequence of SEQ ID NO: 45, and oligonucleotide consisting of the nucleotide sequence of SEQ ID NO: 46.

<Results>

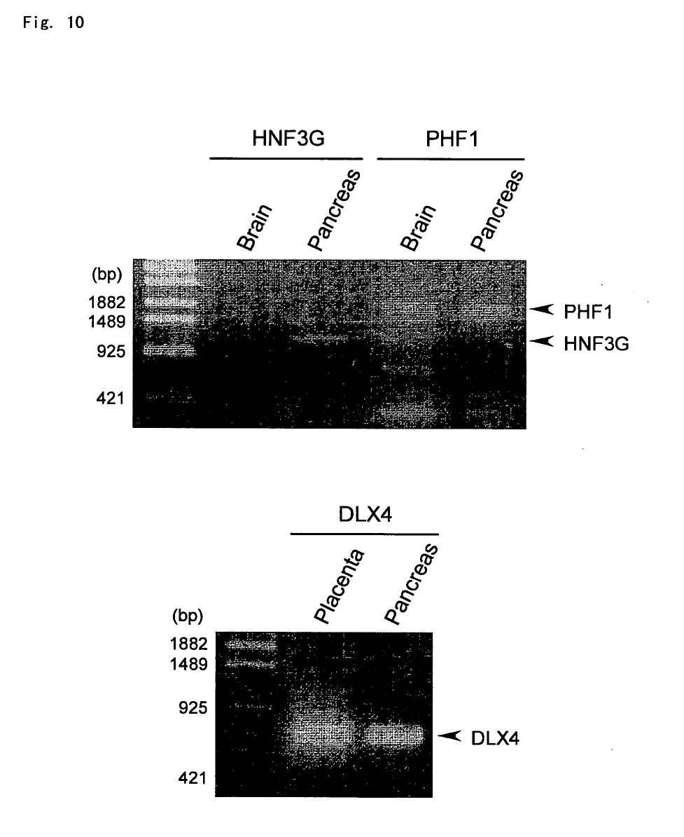

Expressions of HNF3G, PHF1, and DLX4 in the human pancreas as the insulin secreting tissue were examined by RT-PCR. As a result, the presence of mRNA was detected for each gene. FIG. 10 shows the results of PCR using cDNAs derived from the human pancreas (lane of “pancreas”), human brain (lane of “brain”) and human placenta (lane of “placenta”) as templates and primers specific to each gene (panels of “HNF3G,” “PHF1,” and “DLX4,” respectively). Each reaction mixture was separated by 2% agarose gel electrophoresis, and then target PCR products were detected by ethidium bromide staining. The numbers on the left represent values of size markers (bp), and each arrowhead points at the position of the amplification product derived from each mRNA. “Brain,” “Pancreas,” and “Placenta” mean that cDNA derived from each organ was used as a template.

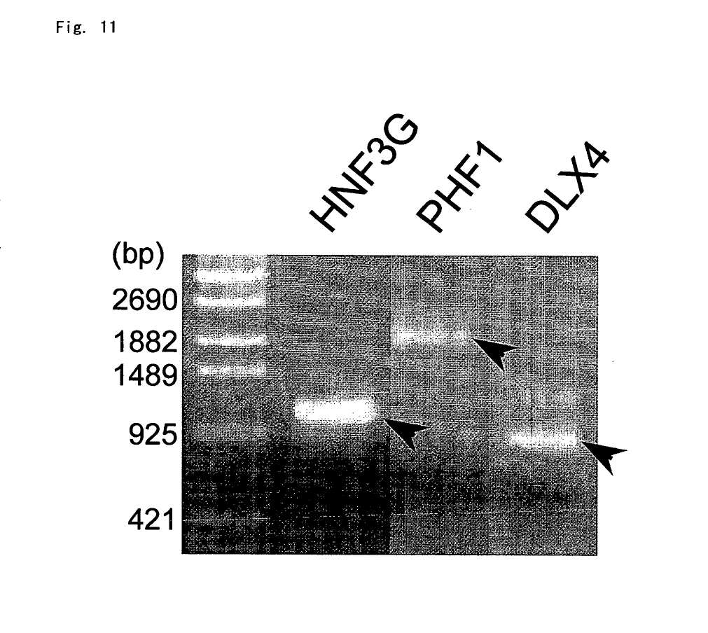

Insulin is secreted from β-cells existing in the pancreas. Expression of each mouse gene in the MIN6 cells, cells of mouse insulinoma derived β-cell strain, was examined by RT-PCR. As a result, the presence of mRNA was detected for each mouse gene. FIG. 11 shows the results of PCR using mouse cDNA derived from MIN6 cells as a template and primers specific to each gene (lanes of “HNF3G,” “PHF1,” and “DLX4,” respectively). Each reaction mixture was separated by 2% agarose gel electrophoresis, and the target PCR products were detected by ethidium bromide staining. The numbers on the left represent values of size markers (bp), and each arrowhead points at the position of the amplification product derived from each mRNA. The primers for detecting each gene were designed so that each of the primers includes intro-exon boundary, and accordingly, the amplified PCR products were not those derived from genomic DNAs.

Sequence Listing Free Text

SEQ ID NO: 13: Partial IPF1 oligopeptide which gave a high score in the local alignment of IPF1 and HNF3G

SEQ ID NO: 14: Partial IPF1 oligopeptide which gave a high score in the local alignment of IPF1 and HNF3G

SEQ ID NO: 15: Partial IPF1 oligopeptide which gave a high score in the local alignment of IPF1 and HNF3G

SEQ ID NO: 16: Partial HNF3G oligopeptide which gave a high score in the local alignment of IPF1 and HNF3G

SEQ ID NO: 17: Partial HNF3G oligopeptide which gave a high score in the local alignment of IPF1 and HNF3G

SEQ ID NO: 18: Partial HNF3G oligopeptide which gave a high score in the local alignment of IPF1 and HNF3G

SEQ ID NO: 19: Partial IPF1 oligopeptide which gave a high score in the local alignment of IPF1 and PHF1

SEQ ID NO: 20: Partial IPF1 oligopeptide which gave a high score in the local alignment of IPF1 and PHF1

SEQ ID NO: 21: Partial PHF1 oligopeptide which gave a high score in the local alignment of IPF1 and PHF1

SEQ ID NO: 22: Partial PHF1 oligopeptide which gave a high score in the local alignment of IPF1 and PHF1

SEQ ID NO: 23: Partial IPF1 oligopeptide which gave a high score in the local alignment of IPF1 and DLX4

SEQ ID NO: 24: Partial IPFL oligopeptide which gave a high score in the local alignment of IPF1 and DLX4

SEQ ID NO: 25: Partial DLX4 oligopeptide which gave a high score in the local alignment of IPF1 and DLX4

SEQ ID NO: 26: Partial DLX4 oligopeptide which gave a high score in the local alignment of IPF1 and DLX4

SEQ ID NO: 27: Partial IPF1 oligopeptide which gave a high score in the local alignment of IPF1 and TCF4

SEQ ID NO: 28: Partial IPF1 oligopeptide which gave a high score in the local alignment of IPF1 and TCF4

SEQ ID NO: 29: Partial IPF1 oligopeptide which gave a high score in the local alignment of IPF1 and TCF4

SEQ ID NO: 30: Partial TCF4 oligopeptide which gave a high score in the local alignment of IPF1 and TCF4

SEQ ID NO: 31: Partial TCF4 oligopeptide which gave a high score in the local alignment of IPF1 and TCF4

SEQ ID NO: 32: Partial TCF4 oligopeptide which gave a high score in the local alignment of IPF1 and TCF4

SEQ ID NO: 33: Partial IPF1 oligopeptide which gave a high score in the local alignment of IPF1 and TMPO

SEQ ID NO: 34: Partial TMPO oligopeptide which gave a high score in the local alignment of IPF1 and TMPO

SEQ ID NO: 35: Primer oligonucleotide designed on the basis of the nucleotide sequence of SEQ ID NO: 3

SEQ ID NO: 36: Primer oligonucleotide designed on the basis of the nucleotide sequence of SEQ ID NO: 3

SEQ ID NO: 37: Primer oligonucleotide designed on the basis of the nucleotide sequence of SEQ ID NO: 5

SEQ ID NO: 38: Primer oligonucleotide designed on the basis of the nucleotide sequence of SEQ ID NO: 5

SEQ ID NO: 39: Primer oligonucleotide designed on the basis of the nucleotide sequence of SEQ ID NO: 7

SEQ ID NO: 40: Primer oligonucleotide designed on the basis of the nucleotide sequence of SEQ ID NO: 7

SEQ ID NO: 41: Primer oligonucleotide designed on the basis of nucleotide sequence of mouse HNF3G gene

SEQ ID NO: 42: Primer oligonucleotide designed on the basis of nucleotide sequence of mouse HNF3G gene

SEQ ID NO: 43: Primer oligonucleotide designed on the basis of nucleotide sequence of mouse PHF1 gene

SEQ ID NO: 44: Primer oligonucleotide designed on the basis of nucleotide sequence of mouse PHF1 gene

SEQ ID NO: 45: Primer oligonucleotide designed on the basis of nucleotide sequence of mouse DLX4 gene

SEQ ID NO: 46: Primer oligonucleotide designed on the basis of nucleotide sequence of mouse DLX4 gene

SEQ ID NO: 47: Partial IPF1 oligopeptide which gave a high score in the local alignment of IPF1 and HNF3G

SEQ ID NO: 48: Partial IPF1 oligopeptide which gave a high score in the local alignment of IPF1 and HNF3G

SEQ ID NO: 49: Partial IPF1 oligopeptide which gave a high score in the local alignment of IPF1 and HNF3G

SEQ ID NO: 50: Partial HNF3G oligopeptide which gave a high score in the local alignment of IPF1 and HNF3G

SEQ ID NO: 51: Partial HNF3G oligopeptide which gave a high score in the local alignment of IPF1 and HNF3G

SEQ ID NO: 52: Partial HNF3G oligopeptide which gave a high score in the local alignment of IPF1 and HNF3G

SEQ ID NO: 53: Partial IPF1 oligopeptide which gave a high score in the local alignment of IPF1 and PHF1

SEQ ID NO: 54: Partial IPF1 oligopeptide which gave a high score in the local alignment of IPF1 and PHF1

SEQ ID NO: 55: Partial PHF1 oligopeptide which gave a high score in the local alignment of IPF1 and PHF1

SEQ ID NO: 56: Partial PHF1 oligopeptide which gave a high score in the local alignment of IPF1 and PHF1

SEQ ID NO: 57: Partial IPF1 oligopeptide which gave a high score in the local alignment of IPF1 and DLX4

SEQ ID NO: 58: Partial IPF1 oligopeptide which gave a high score in the local alignment of IPF1 and DLX4

SEQ ID NO: 59: Partial IPF1 oligopeptide which gave a high score in the local alignment of IPF1 and DLX4

SEQ ID NO: 60: Partial DLX4 oligopeptide which gave a high score in the local alignment of IPF1 and DLX4

SEQ ID NO: 61: Partial DLX4 oligopeptide which gave a high score in the local alignment of IPF1 and DLX4

SEQ ID NO: 62: Partial DLX4 oligopeptide which gave a high score in the local alignment of IPF1 and DLX4

SEQ ID NO: 63: Partial IPF1 oligopeptide which gave a high score in the local alignment of IPF1 and TCF4

SEQ ID NO: 64: Partial IPF1 oligopeptide which gave a high score in the local alignment of IPF1 and TCF4

SEQ ID NO: 65: Partial IPF1 oligopeptide which gave a high score in the local alignment of IPF1 and TCF4

SEQ ID NO: 66: Partial TCF4 oligopeptide which gave a high score in the local alignment of IPF1 and TCF4

SEQ ID NO: 67: Partial TCF4 oligopeptide which gave a high score in the local alignment of IPF1 and TCF4

SEQ ID NO: 68: Partial TCF4 oligopeptide which gave a high score in the local alignment of IPF1 and TCF4

SEQ ID NO: 69: Partial IPF1 oligopeptide which gave a high score in the local alignment of IPF1 and TMPO

SEQ ID NO: 70: Partial TMPO oligopeptide which gave a high score in the local alignment of IPF1 and TMPO

### INDUSTRIAL APPLICABILITY

The present invention provides the proteins (i) to (v) that bind to IPF1, and also provides a means for regulating the insulin gene transcription based on the inhibition of the binding of IPF1 and the aforementioned proteins. By the screening method of the present invention using this means, for example, a substance promoting the insulin gene transcription can be easily screened, and the resulting substance can be used as an active ingredient of a medicament for prophylactic and/or therapeutic treatment of diseases such as diabetes.

1. A method for promoting insulin gene transcription, which comprises the step of inhibiting binding of IPF1 and any one of proteins selected from the following group:
(i) HNF3G,
(ii) PHF1,
and
(iii) DLX4.

2. A method for screening a substance that inhibits binding of IPF1 and any one of proteins selected from the following group, which comprises the step of bringing a test substance into contact with IPF1 and/or said protein under a condition that allows the binding of IPF1 and said protein and then determining whether or not the test substance inhibits the binding of IPF1 and said protein by detecting presence or absence, or change of a signal and/or a marker generated by the binding of IPF1 and said protein in a system in which the signal and/or the marker can be detected:
(i) HNF3G,
(ii) PHF1,
(iii) DLX4,
(iv) TCF4,
and
(v) TMPO.

3. A method for screening a substance that promotes insulin gene transcription, which comprises the step of bringing a test substance into contact with IPF1 and/or any one of proteins selected from the following group under a condition that allows the binding of IPF1 and said protein and then determining whether or not the test substance inhibits the binding of IPF1 and said protein by detecting presence or absence, or change of a signal and/or a marker generated by the binding of IPF1 and said protein in a system in which the signal and/or the marker can be detected:
(i) HNF3G,
(ii) PHF1,
and
(iii) DLX4.

4. A method for screening a substance that promotes insulin gene transcription, which comprises the step of bringing a test substance into contact with IPF1 and/or any one of proteins selected from the following group under a condition that allows the binding of IPF1 and said protein to determine whether or not insulin gene transcription is promoted:
(i) HNF3G,
(ii) PHF1,
and
(iii) DLX4.

5. A substance screened by the screening method according to claim 2 any one of claims 2.

6. A medicament for prophylactic and/or therapeutic treatment of a disease caused by a reduced amount of a gene product of insulin gene, which inhibits binding of IPF1 and any one of proteins selected from the following group:
(i) HNF3G,
(ii) PHF1,
and
(iii) DLX4.

7. A medicament for prophylactic and/or therapeutic treatment of diabetes, which inhibits binding of IPF1 and any one of proteins selected from the following group:
(i) HNF3G,
(ii) PHF1,
and
(iii) DLX4.

8. The medicament according to claim 6 or 7, which contains a substance screened by a screening method that screens a substance that inhibits binding of IPF1 and any one of proteins selected from the following group, which comprises the step of bringing a test substance into contact with IPF1 and/or said protein under a condition that allows the binding of IPF1 and said protein and then determining whether or not the test substance inhibits the binding of IPF1 and said protein by detecting presence or absence, or chance of a signal and/or a marker generated by the binding of IPF1 and said protein in a system in which the signal and/or the marker can be detected:
(i) HNF3G,
(ii) PHF1,
(iii) DLX4,
(iv) TCF4,
and
(v) TMPO as an active ingredient.

9. A method for prophylactic and/or therapeutic treatment of a disease caused by a reduced amount of a gene product of insulin gene, which comprises the step of inhibiting binding of IPF1 and any one of proteins selected from the following group:
(i) HNF3G,
(ii) PHF1,
and
(iii) DLX4.

10. A method for prophylactic and/or therapeutic treatment of diabetes, which comprises the step of inhibiting binding of IPF1 and any one of proteins selected from the following group:
(i) HNF3G,
(ii) PHF1,
and
(iii) DLX4.

11. The method according to claim 9 or 10, which comprises the step of administering an effective amount of a substance screened by a screening method that screens a substance that inhibits binding of IPF1 and any one of proteins selected from the following group, which comprises the step of bringing a test substance into contact with IPF1 and/or said protein under a condition that allows the binding of IPF1 and said protein and then determining whether or not the test substance inhibits the binding of IPF1 and said Protein by detecting presence or absence, or chance of a signal and/or a marker generated by the binding of IPF1 and said protein in a system in which the signal and/or the marker can be detected:
(i) HNF3G,
(ii) PHF1,
(iii) DLX4,
(iv) TCF4,
and
(v) TMPO to a mammal including a human.

12. A kit of reagents used in the screening method according to claim 2, which comprises:
(a) IPF1 and/or DNA coding for IPF1, and
(b) one or more kinds of proteins selected from the following group and/or DNAs encoding the proteins:
(i) HNF3G,
(ii) PHF1,
and
(iii) DLX4.
